(19) United States
(12) Patent Application Publication
(10) Pub. No.: US 2012/0245128 A1
(43) Pub. Date: Sep. 27, 2012

(54) RAPID DETECTION AND QUANTIFICATION OF MODIFICATION OF MEDICINAL COMPOUNDS AND DRUG RESISTANCE ACTIVITY

(75) Inventors: Anthony M. Haag, Houston, TX (US); Norbert K. Herzog, Friendswood, TX (US); David W. Niesel, Friendswood, TX (US)

(73) Assignee: The Board of Regents of the University of Texas System, Austin, TX (US)

(21) Appl. No.: 13/429,594

(22) Filed: Mar. 26, 2012

Related U.S. Application Data
(60) Provisional application No. 61/467,709, filed on Mar. 25, 2011.

(51) Int. Cl. G01N 27/62; A61K 31/337; A61K 31/675; A61K 31/431; A61K 31/546

(52) U.S. Cl. 514/90; 435/32; 514/196; 514/206; 514/449

(57) ABSTRACT

The present disclosure in general relates to methods, systems, and apparatus for identifying modification of a medicinal compound exposed to a sample for use in determining which treatment to provide to a subject in need thereof.

This application claims priority to U.S. Provisional Patent Application Ser. No. 61/467,709 filed Mar. 25, 2011, which is incorporated herein by reference in its entirety.

### STATEMENT REGARDING FEDERALLY FUNDED RESEARCH

None

### REFERENCE TO SEQUENCE LISTING

None.

### BACKGROUND

The field of the invention is detection of modified medicinal compounds and drug resistance, including antibiotic resistance and chemotherapeutic resistance.

Antibiotics kill or inhibit the growth of bacteria. The main classes of antibiotics are penicillins, cephalosporins, macrolides, fluoroquinolones, sulfonamides, tetracyclines, and aminoglycosides. A given bacterial strain may be only susceptible to certain antibiotics. Bacteria develop ways to become resistant to an antibiotic. In an example, when antibiotics are used, the bacteria sensitive to the antibiotic are killed, while those that are resistant survive. This is called selective pressure. A given bacteria may survive because it has developed a mechanism to resist the effects of the antibiotic. The surviving bacteria therefore continue to multiply. Bacteria may gain resistance through mutation or acquiring DNA from other bacteria (transposons) that codes for resistance to a particular antibiotic. Multi-antibiotic resistant bacteria can arise when transposons for antibiotic resistance transfer from chromosomal DNA, to plasmid DNA, and into the chromosomal DNA of another bacterium. An infection due to multi-antibiotic resistant bacteria may be hard to treat because of the lack of antibiotics that are effective against the bacteria.

Bacteria resist the effects of antibiotics by several different methods. Bacteria may prevent the antibiotic from entering the cell or use pumps that move the antibiotic out of the cell quickly enough to prevent the adverse affects. Bacteria may also develop mutations in the target of the antibiotic that no longer allow the antibiotic to interact with its intended target, rendering the antibiotic ineffective. Bacteria also utilize enzymes that actively disable the antibiotic by modifying its structure, such as by β-lactamase and chloramphenicol acetyl-transferase.

Overuse of antibiotics along with the adaptability of the bacteria on which the antibiotics are used has created a growing number of resistant strains of these bacteria. Clinical treatment of an infection with ineffective antibiotics can lead to increased bacterial growth and spread in addition to serious medical complications. Newly resistant strains can emerge by genetic mutation as a result of overuse of antibiotics that causes selection for these resistant forms. This inhibits the future effectiveness of those antibiotics.

Timely detection of antibiotic resistance is an issue of growing concern as more resistant and multi-resistant bacterial strains emerge. Currently used phenotypic tests are effective, but usually take at least one day and many times require a purified bacterial population. Molecular methods are more rapid, but due to cost, molecular methods are not a viable option for large scale, frequent use. Detection of the antibiotics to which a bacterium is resistant is key in designing a treatment plan and in minimizing the chances of increasing antibiotic resistance in a bacterial population.

Cancer cells can also be inherently resistant to the chemotherapeutics or can acquire resistance through several means including: expression of transporters that eject the chemotherapeutic drugs, insensitivity to drug induced apoptosis, mutations in drug targets, and the chemical modification by cellular enzymes that inactivate the drugs and/or target them for export. The issue of single and multiply drug resistant tumors has become even more serious with the recognition of the presence of tumorigenic stem cells in a variety of tumors. Detection of the chemotherapeutics to which tumor cells are resistant is key in designing a treatment plan.

### SUMMARY

Certain embodiments are directed to methods of identifying a drug resistance profile of a sample comprising one or more of the following steps: exposing a sample to at least one drug; conducting mass spectrometry on at least one drug after exposure to the sample and analyzing mass to charge ratio of ions from the at least one drug; and detecting the presence of one or more ions indicative of the drug and/or a metabolite of the drug. In certain aspects, the mass spectrometry is electrospray mass spectrometry. In a further aspect, the mass spectrometry is selected reaction monitoring. The sample can comprise at least one antibiotic resistant bacterium. In certain aspects, the sample comprises 1, 2, 3, 4, 5, 6, 7, 8, 9, 10 or more bacteria. In certain aspects, the sample comprises tumor cells. In a further aspect, the sample is exposed to more than one drug. In still a further aspect, the sample is exposed to 1, 2, 3, 4, 5, 6, 7, 8, 9, 10 or more drugs. The methods can include detecting ions from more than one drug. The drugs can be detected simultaneously or at different times. Drugs and their related ions can be detected on the same or different apparatus.

The drug can be an antibiotic. In certain embodiments, the antibiotic is a member of the penicillin or cephalosporin family of antibiotics. The penicillin family includes, but is not limited to ampicillin, amoxicillin, azlocillin, bacampicillin, cefixime, carbenicillin, methicillin, cloxacillin, 6-APA, piperacillin, pivmecillinam, penicillin V, monolactam, aztreonam, mecillinam, imipenem, and meropenem. The cephalosporin family includes, but is not limited to cefoperazone, latamoxef, cephapirin, cefazolin, cefaclor, ceftibuten, ceftizoxime, cefotetan, cefuroxime, cefprozil, ceftazidime, cephaloglycine, cephaloridine, nitrocephine, cefatoxime, ceftiofur, cephapyrine, cefepime, cefpirome, cefadroxil, cefamandole, cefoxitin, cefpodoxime, ceftriaxone, cephalexin, cephazoline, cephradine and 7-ACA.

In certain aspects, drug is a chemotherapeutic agent. Chemotherapeutic agents include, but are not limited to cyclophosphamide and paclitaxel.

In certain aspects, the time between exposing a sample to a drug and conducting mass spectrometry is less than 5, 10, 15, 20, 25, 30, 35, 40, 45, 50, 55, 60, or 120 minutes.

The methods described herein can detect one or more ions that are indicative of a metabolite of the drug or inactivation of a drug. In certain aspects, metabolites can be a glucoronidation, sulfation, oxidation, hydroxylation, dealkylation, or hydrolysis product. IN further aspects, the metabolite is a hydrolysis product.

The methods described herein can further comprise administering a drug to a patient, from which the sample was obtained, that is not inactivated upon exposure to the sample. In certain aspects, a drug is identified to which the patient or infecting microbes are not resistant or are susceptible to.

In certain aspects, the sample is selected from the group consisting of sputum, saliva, urine, stool, spinal fluid, lung lavage, intestinal lavage, nasopharyngeal lavage and blood.

Disclosed herein are methods of identifying resistance to one or more antibiotics by bacteria present in a sample comprising one or more of the following steps: obtaining a sample that may comprise bacteria; exposing the sample to one or more antibiotics; analyzing mass to charge ratio of the one or more antibiotics that have not been exposed to the sample; analyzing mass to charge ratio of the one or more antibiotics exposed to the sample; comparing the mass to charge ratio of the one or more antibiotics exposed to the sample to the mass to charge ratio of the one or more antibiotics that have not been exposed to the sample; and identifying resistance to the one or more antibiotics by bacteria present in the sample if the mass to charge ratio of the one or more antibiotics exposed to the sample is different than the mass to charge ratio of the one or more antibiotics not exposed to the sample. Multiple strains of the bacteria may be resistant to one or more antibiotics. The mass to charge ratio of more than one antibiotic may be analyzed. Resistance to more than one antibiotic may be identified. The mass to charge ratio may be analyzed using mass spectrometry. Selected reaction monitoring (SRM) may be utilized to analyze the mass to charge ratio.

Disclosed herein are methods of identifying resistance to one or more antibiotics by bacteria present in a sample comprising obtaining a sample that may comprise bacteria; exposing the sample to one or more antibiotics; analyzing mass to charge ratio of the one or more antibiotics exposed to the sample; analyzing mass to charge ratio of the one or more antibiotics exposed to a bacteria known to be resistant to the one or more antibiotics; comparing the mass to charge ratio of the one or more antibiotics exposed to the sample to the mass to charge ratio of the one or more antibiotics exposed to a bacteria known to be resistant to the one or more antibiotics; and identifying resistance to the one or more antibiotics by bacteria present in the sample if the mass to charge ratio of the one or more antibiotics exposed to the sample is similar to the mass to charge ratio of the one or more antibiotics exposed to a bacteria known to be resistant to that antibiotic. Multiple strains of the bacteria may be resistant to one or more antibiotics. The mass to charge ratio of more than one antibiotic may be analyzed. Resistance to more than one antibiotic may be identified. The mass to charge ratio may be analyzed using mass spectrometry.

Disclosed herein are methods of treatment comprising obtaining a sample from a patient; exposing the sample to one or more antibiotics; analyzing mass to charge ratio of the one or more antibiotics that have not been exposed to the sample; analyzing mass to charge ratio of the one or more antibiotics exposed to the sample; comparing the mass to charge ratio of the one or more antibiotics exposed to the sample to the mass to charge ratio of the one or more antibiotics that have not been exposed to the sample; identifying resistance to the one or more antibiotics by bacteria present in the sample if the mass to charge ratio of the one or more antibiotics exposed to the sample is different than the mass to charge ratio of the one or more antibiotics not exposed to the sample; and administering to the patient one or more antibiotics to which the bacteria present in the sample are not resistant. The method may further comprise not administering to the patient one or more antibiotics to which bacteria present in the sample are resistant.

Disclosed herein are methods of determining the identity of an antibiotic resistant bacteria in a sample comprising obtaining a sample; exposing the sample to one or more antibiotics; analyzing mass to charge ratio of the one or more antibiotics that have not been exposed to the sample; analyzing mass to charge ratio of the one or more antibiotics exposed to the sample; comparing the mass to charge ratio of the one or more antibiotics exposed to the sample to the mass to charge ratio of the one or more antibiotics that have not been exposed to the sample; identifying resistance to the one or more antibiotics by bacteria present in the sample if the mass to charge ratio of the one or more antibiotics exposed to the sample is different than the mass to charge ratio of the one or more antibiotics not exposed to the sample; and determining the identity of an antibiotic resistant bacteria by comparing it to a known pattern of which antibiotics the antibiotic resistant bacteria is resistant and non-resistant.

Disclosed herein are methods of testing multiple strains of bacteria in a sample for antibiotic resistance comprising obtaining a sample; exposing the sample to one or more antibiotics; analyzing mass to charge ratio of the one or more antibiotics that have not been exposed to the sample; analyzing mass to charge ratio of the one or more antibiotics exposed to the sample; comparing the mass to charge ratio of the one or more antibiotics exposed to the sample to the mass to charge ratio of the one or more antibiotics that have not been exposed to the sample; and identifying resistance to the one or more antibiotics by multiple strains of bacteria present in the sample if the mass to charge ratio of the one or more antibiotics exposed to the sample is different than the mass to charge ratio of the one or more antibiotics not exposed to the sample. The multiple strains of bacteria may be tested for resistance to more than one antibiotic.

Disclosed herein are methods of determining to which antibiotics bacteria present in a sample are not resistant to comprising obtaining a sample; exposing the sample to one or more antibiotics; analyzing mass to charge ratio of the one or more antibiotics that have not been exposed to the sample; analyzing mass to charge ratio of the one or more antibiotics exposed to the sample; comparing the mass to charge ratio of the one or more antibiotics exposed to the sample to the mass to charge ratio of the one or more antibiotics that have not been exposed to the sample; and identifying that bacteria present in a sample are not resistant to the one or more antibiotics if the mass to charge ratio of the one or more antibiotics exposed to the sample is similar to the mass to charge ratio of the one or more antibiotics not exposed to the sample.

Disclosed herein are microfluidic devices for detecting antibiotic resistance comprising a solid substrate comprising a sample entry port, buffer in a buffer chamber, and an antibiotic chamber, wherein upon adding a sample to the microfluidic device, the sample, the buffer, and the antibiotic mix together, providing an output sample to be injected for analysis of the mass to charge ratio of the antibiotic. A chamber may be present to separate the antibiotic based upon molecular weight. The output sample may be analyzed by HPLC. The output sample may be analyzed by mass spectrometry. The microfluidic device may lyse cells present in the sample.

Disclosed herein are systems for detecting antibiotic resistant bacteria in a sample comprising a sample to be analyzed for the presence of bacteria resistant to one or more antibiotics; a mass spectrometer wherein the mass spectrometer provides a test spectra of one or more antibiotics following exposure of the one or more antibiotics to the sample; and a computer linked to the mass spectrometer containing files of standard spectra of one or more antibiotics not exposed to the sample, wherein comparison of the test spectra and the standard spectra indicate that antibiotic resistant bacteria are present in the sample if a peak for the antibiotic in the test spectra is decreased in relation to the size of the peak of the antibiotic in the standard spectra. Multiple strains of bacteria may be present in the sample. The mass to charge ratio of more than one antibiotic may be analyzed. Resistance to more than one antibiotic may be identified.

Disclosed herein are apparatus for detecting antibiotic resistant bacteria in a sample comprising a mass spectrometer wherein the mass spectrometer provides a test spectra of one or more antibiotics following exposure of the antibiotic to a sample; and a computer linked to the mass spectrometer containing files of standard spectra of one or more antibiotics not exposed to the sample, wherein comparison of the test spectra and the standard spectra indicate that antibiotic resistant bacteria are present in the sample if a peak at a mass to charge ratio for the one or more antibiotics in the test spectra is decreased in relation to the size of the peak at a mass to charge ratio of the one or more antibiotics in the standard spectra.

Disclosed herein are methods of identifying modification of a medicinal compound present in a sample comprising obtaining a sample from a patient; exposing the sample to one or more medicinal compounds; analyzing mass to charge ratio of the medicinal compound that was not present in the sample; analyzing mass to charge ratio of the medicinal compound present in the sample; comparing the mass to charge ratio of the medicinal compound present in the sample to the mass to charge ratio of the medicinal compound that was not present in the sample; and identifying modification of the medicinal compound present in the sample if the mass to charge ratio of the medicinal compound present in the sample is different than the mass to charge ratio of the medicinal compound that was not present in the sample.

Disclosed herein are microfluidic devices for identifying modification of a medicinal compound present in a sample comprising a solid substrate comprising a sample entry port, buffer in a buffer chamber, and an medicinal compound chamber, wherein upon adding a sample to the microfluidic device, the sample, the buffer, and the medicinal compound mix together, providing an output sample to be injected for analysis of the mass to charge ratio of the medicinal compound. A chamber may be present to separate the medicinal compound based upon molecular weight. The output sample may be analyzed by HPLC. The output sample may be analyzed by mass spectrometry. The microfluidic device may lyse cells present in the sample.

Disclosed herein are methods of identifying resistance of tumor cells to a chemotherapeutic exposed to a sample comprising obtaining a sample that may comprise tumor cells resistant to a chemotherapeutic; exposing the sample to one or more chemotherapeutics; analyzing mass to charge ratio of the chemotherapeutic that was not exposed to the sample; analyzing mass to charge ratio of the chemotherapeutic that was exposed to the sample; comparing the mass to charge ratio of the chemotherapeutic exposed to the sample to the mass to charge ratio of the chemotherapeutic that was not exposed to the sample; and identifying resistance to the chemotherapeutic exposed to the sample if the mass to charge ratio of the chemotherapeutic exposed to the sample is different than the mass to charge ratio of the chemotherapeutic that was not exposed to the sample. The chemotherapeutic may be cyclophosphamide. The chemotherapeutic may be paclitaxel.

Disclosed herein are microfluidic devices for identifying resistance of tumor cells to a chemotherapeutic exposed to a sample that may comprise tumor cells resistant to a chemotherapeutic comprising a solid substrate comprising a sample entry port, buffer in a buffer chamber, and a chemotherapeutic chamber, wherein upon adding a sample to the microfluidic device, the sample, the buffer, and the chemotherapeutic mix together, providing an output sample to be injected for analysis of the mass to charge ratio of the chemotherapeutic. A chamber may be present to separate the medicinal compound based upon molecular weight. The output sample may be analyzed by HPLC. The output sample may be analyzed by mass spectrometry. The microfluidic device may lyse tumor cells present in the sample.

Disclosed herein are systems for detecting resistance tumor cells to a medicinal compound in a sample comprising a sample to be analyzed to detect resistance of a tumor cell to a chemotherapeutic; a mass spectrometer wherein the mass spectrometer provides a test spectra of one or more chemotherapeutics following exposure to the sample; and a computer linked to the mass spectrometer containing files of standard spectra of one or more chemotherapeutics not exposed to the sample, wherein comparison of the test spectra and the standard spectra indicate that the tumor cells are resistant to the chemotherapeutic if a peak for the chemotherapeutic in the test spectra is decreased in relation to the size of the peak of the chemotherapeutic in the standard spectra. The mass to charge ratio of more than one chemotherapeutic may be analyzed. Resistance of tumor cells to more than one chemotherapeutic may be identified.

Disclosed herein are apparatus for detecting resistance of tumor cells to a medicinal compound in a sample comprising a mass spectrometer wherein the mass spectrometer provides a test spectra of one or more chemotherapeutics following exposure of the chemotherapeutics to a sample to be analyzed to detect resistance of a tumor cell to a medicinal compound; and a computer linked to the mass spectrometer containing files of standard spectra of one or more chemotherapeutics not exposed to the sample, wherein comparison of the test spectra and the standard spectra indicate resistance of the tumor cells to the chemotherapeutics if a peak at a mass to charge ratio for the one or more chemotherapeutics in the test spectra is decreased in relation to the size of the peak at a mass to charge ratio of the one or more chemotherapeutics in the standard spectra.

Other embodiments of the invention are discussed throughout this application. Any embodiment discussed with respect to one aspect of the invention applies to other aspects of the invention as well and vice versa. Each embodiment described herein is understood to be embodiments of the invention that are applicable to all aspects of the invention. It is contemplated that any embodiment discussed herein can be implemented with respect to any method or composition of the invention, and vice versa. Furthermore, compositions and kits of the invention can be used to achieve methods of the invention.

The use of the word “a” or “an” when used in conjunction with the term “comprising” in the claims and/or the specification may mean “one,” but it is also consistent with the meaning of “one or more,” “at least one,” and “one or more than one.”

Throughout this application, the term “about” is used to indicate that a value includes the standard deviation of error for the device or method being employed to determine the value.

The use of the term “or” in the claims is used to mean “and/or” unless explicitly indicated to refer to alternatives only or the alternatives are mutually exclusive, although the disclosure supports a definition that refers to only alternatives and “and/or.”

As used in this specification and claim(s), the words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) are inclusive or open-ended and do not exclude additional, unrecited elements or method steps.

Other objects, features and advantages of the present invention will become apparent from the following detailed description. It should be understood, however, that the detailed description and the specific examples, while indicating specific embodiments of the invention, are given by way of illustration only, since various changes and modifications within the spirit and scope of the invention will become apparent to those skilled in the art from this detailed description.

### DESCRIPTION OF THE DRAWINGS

The following drawings form part of the present specification and are included to further demonstrate certain aspects of the present invention. The invention may be better understood by reference to one or more of these drawings in combination with the detailed description of the specification embodiments presented herein.

FIG. 1 depicts the hydrolyzation of a lactam ring by β-lactamase.

FIG. 2 depicts the acetylation of chloramphenicol by chloramphenicol acetyltransferase.

FIG. 3 depicts the process of selected reaction monitoring (SRM).

FIG. 4 depicts degradation of ampicillin to ampicillin penicilloic acid by β-lactamase.

FIG. 5 depicts SRM of ampicillin.

FIG. 6A depicts a base peak chromatogram of ampicillin resistant E. coli harboring pUC18.

FIG. 6B depicts an electrospray mass spectrum at retention time 37 minutes of a media sample from ampicillin resistant E. coli harboring pUC18.

FIG. 7A depicts a base peak chromatogram of non-ampicillin resistant E. coli RR1.

FIG. 7B depicts an electrospray mass spectrum at retention time 37 minutes of a media sample from non-ampicillin resistant E. coli RR1.

FIG. 7C depicts an electrospray mass spectrum at retention time 35 minutes of a media sample from non-ampicillin resistant E. coli RR1.

FIG. 8 depicts a spectra of hydrolyzed ampicillin.

FIG. 9 depicts SRM of a media sample from non-ampicillin resistant E. coli RR1 and ampicillin resistant E. coli.

FIG. 10 depicts a base peak chromatogram of ampicillin incubated with β-lactamase.

FIG. 11A depicts the hydrolysis of ampicillin by β-lactamase and the carboxylic acid formed via the β-lactam ring reacting with the amine group of another ampicillin molecule through an electrophilic reaction with the dimer being hydrolyzed again by β-lactamase.

FIG. 11B depicts the formation of the ampicillin product ion of (m/z=160.1) after collision induced dissociation (CID).

FIG. 12A depicts the SRM of ampicillin (m/z 350.1 to 160.1).

FIG. 12B depicts the SRM of the formation of hydrolyzed ampicillin (m/z 359.1 to 160.1).

FIG. 13 depicts the SRM of a media sample from chloramphenicol resistant E. coli and chloramphenicol susceptible E. coli.

FIG. 14 depicts the loss of the hydroxyls and a methylene group in chloramphenicol during CID.

FIG. 15 depicts two possible acetylation sites for chloramphenicol with the same m/z value (365.0) for the precursor ion and the same CID product ion (275.0).

FIG. 16 depicts the reduction of chloramphenicol in a broth over time.

FIG. 17 depicts the formation of the inactive acetylchloramphenicol over the same period of time.

FIG. 18A depicts a spectra of a five antibiotic mixture of ampicillin, piperacillin, cephalexin, cloxacillin, and chloramphenicol.

FIG. 18B depicts a spectra of a five antibiotic mixture of ampicillin, piperacillin, cephalexin, cloxacillin, and chloramphenicol after being subjected to β-lactamase for less than 5 minutes.

FIG. 19 depicts the number of bacteria detectable in a 1/25 assay mix in dilution buffer.

FIG. 20 depicts the number of bacteria harboring pACYC184 (chloramphenicol resistant) and pUC 18 (ampicillin resistant) detectable in an undiluted assay mix.

FIG. 21A is a profile of ampicillin.

FIG. 21B is a profile of ampicillin after being exposed to β-lactamase using column and mass spectrometry.

FIG. 22A is a profile of cloxacillin.

FIG. 22B is a profile of cloxacillin after being exposed to β-lactamase.

### DESCRIPTION

The disclosure relates to the detection of antibiotic resistance. It will be appreciated that for simplicity and clarity of illustration, where considered appropriate, reference numerals may be repeated among the figures to indicate corresponding or analogous elements. In addition, numerous specific details are set forth in order to provide a thorough understanding of the example embodiments described herein. However, it will be understood by those of ordinary skill in the art that the example embodiments described herein may be practiced without these specific details. In other instances, methods, procedures and components have not been described in detail so as not to obscure the embodiments described herein.

The term “mass to charge ratio” when used in this specification and claims, refers to mass being the molecular weight and charge being the charge of a molecule after ionization with the ratio being the molecular weight divided by the charge. Mass to charge ratio is also written as m/z.

The term “SRM”, when used in this specification and claims, refers to selected reaction monitoring. In selected reaction monitoring (SRM), two mass analyzers act as mass filters to monitor a product ion of a selected precursor ion. The detector provides an intensity value over time by counting the ion matching the selected transition. The two m/z values associated with the precursor and product ion are called a “transition” are written as precursor m/z>product m/z.

The term “HPLC”, when used in this specification and claims, refers to high performance liquid chromatography or high-pressure liquid chromatography. In HPLC, a mobile phase is forced under pressure through a stationary phase.

The term “mass spectrometry”, when used in this specification and claims, refers to a method to identify compounds by their mass or mass-to-charge ratio (m/z).

The term “similar to”, when used in this specification and claims, refers to being marked by correspondence or resemblance.

The term “β-lactam antibiotic”, when used in this specification and claims, refers to penicillins and cephalosporins.

The term “penicillins”, when used in this specification and claims, refers to including but not limited to ampicillin, amoxicillin, azlocillin, bacampicillin, cefixime, carbenicillin, methicillin, cloxacillin, 6-APA, piperacillin, pivmecillinam, penicillin V, monolactam, aztreonam, mecillinam, imipenem, and meropenem.

The term “cephalosporins”, when used in this specification and claims, refers to including but not limited to cefoperazone, latamoxef, cephapirin, cefazolin, cefaclor, ceftibuten, ceftizoxime, cefotetan, cefuroxime, cefprozil, ceftazidime, cephaloglycine, cephaloridine, nitrocephine, cefatoxime, ceftiofur, cephapyrine, cefepime, cefpirome, cefadroxil, cefamandole, cefoxitin, cefpodoxime, ceftriaxone, cephalexin, cephazoline, cephradine and 7-ACA.

The term “medium”, when used in this specification and claims, refers to liquid, gel, or semi-solid material designed to support the growth of bacteria.

The term “medicinal compound”, when used in this specification and claims, refers to including but not limited to a substance that treats, prevents, or alleviates symptoms of a condition.

The overall goal of the assay disclosed herein is to be able to rapidly identify strains of bacteria that are resistant to a variety of antibiotics.

Various assays are disclosed herein for the rapid detection of antibiotic resistant bacteria. The assay is a rapid, reliable, relatively inexpensive method to detect antibiotic resistant bacteria. In an example, the assay is mass spectrometry based. Mass spectrometry allows higher resolution and more rapid detection than do traditional culture methods. Mass spectrometric analysis of culture media supernatants from antibiotic resistant and non-resistant bacteria show distinct spectra of the antibiotic and breakdown products of the antibiotic that provide an easily reproducible “fingerprint”.

Bacteria have developed several methods to resist the effects of antibiotics. Bacteria may prevent the antibiotic from entering the cell or use pumps that move the antibiotic out of the cell quickly enough to prevent the adverse affects. Bacteria may also develop mutations in the target of the antibiotic that do not allow the antibiotic to interact with its intended target. Some bacteria use enzymes to actively modify the structure and disable the antibiotic, such as β-lactamase and chloramphenicol acetyl-transferase.

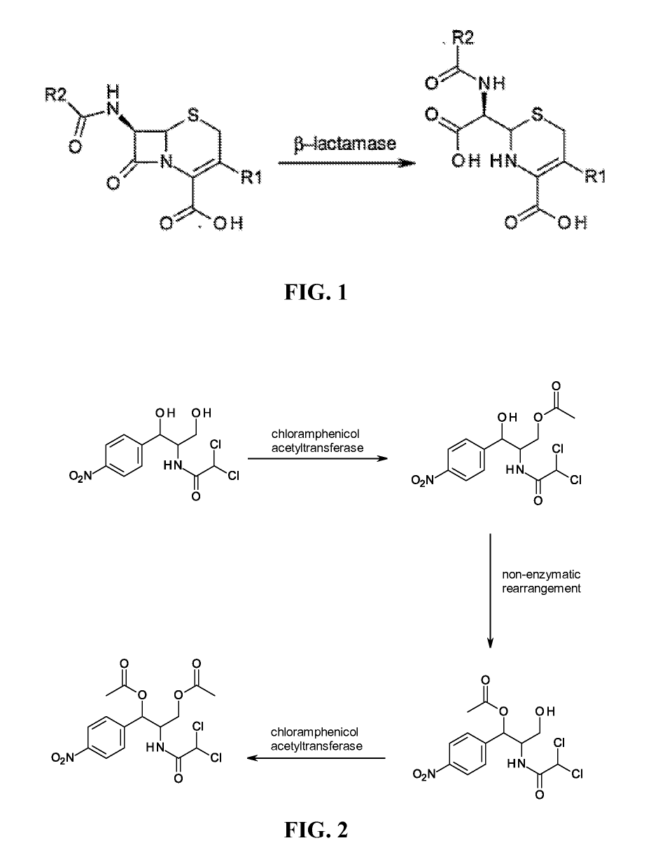

Penicillins and cephalosporins kill bacteria by inhibiting the synthesis of the peptidoglycan layer in the bacterial cell wall. The β-lactam group of a penicillin or cephalosporin binds to transpeptidases, enzymes that link the peptidoglycans in the bacterial cell wall. In the case of penicillins and cephalosporins, resistant organisms express β-lactamase that hydrolyzes the lactam ring of both penicillins and cephalosporins (FIG. 1). After cleavage of the β-lactam ring, the antibiotic is not able to bind to the transpeptidases.

Chloramphenicol inhibits growth of bacteria by inhibiting the protein synthesis of the bacteria by binding to the 50S ribosome and inhibiting peptidyl transferase activity. Chloramphenicol resistant organisms express chloramphenicol acetyltransferase that attaches an acetyl group to chloramphenicol, preventing chloramphenicol from binding to ribosomes. (FIG. 2).

Carbapenems are a class of β-lactam antibiotics that prevent linking of the peptidoglycan strands and synthesis of the bacterial cell wall by binding to penicillin-binding proteins (PBP). PBPs are involved in the final stages of peptidoglycan synthesis of cell walls. Bacteria may exhibit resistance to carbapenems by expression of carbapenemase. Carbapenemase can hydrolyze penicillins, cephalosporins, monolactams, and carbapenems.

Aminoglycoside antibiotics kill bacteria by inhibiting protein synthesis of the bacteria by irreversibly binding to the 30S ribosomal subunit and freezing the 30S initiation complex. Bacteria may exhibit resistance to aminoglycosides by phosphorylation, adenylylation, or acetylation of the aminoglycoside. Phosphorylation, and subsequent inactivation, of the aminoglycoside occurs by aminoglycoside kinase.

Tetracycline antibiotics inhibit the growth of bacteria by inhibiting protein synthesis of the bacteria by reversibly binding to the 30S ribosomal subunit and inhibiting binding of the aminoacyl-tRNA to the 70S ribosome. Resistance to tetracycline may occur by chemical modification of tetracycline or by other methods such as pumping the tetracycline out of the cell. TetX is a FAD-dependent monooxygenase that hydroxylates tetracycline. Hydroxylated tetracycline is unstable and rapidly decomposes.

Macrolide antibiotics inhibit the growth of bacteria by reversibly binding to the P site on the 50S subunit of the ribosome. Macrolides are inactivated by bacteria expressing enzymes such as erythromycin esterases or macrolide 2′ phosphotransferase. Erythromycin esterases inactive the lactone ring in 14-membered ring macrolides. Macrolide 2′ phosphotransferases add a phosphate to the 2′-hydroxyl group of macrolides.

Quinolones kill bacteria by inhibiting DNA synthesis by binding to the A subunit of the DNA gyrase and preventing supercoiling of DNA. Bacteria decrease the potency of quinolones by expressing a mutant of aminoglycoside acetyltransferase that acetylates quinolones that have an unsubstituted amino nitrogen on the piperazinyl moiety.

Sulfonamides inhibit the growth of bacteria by inhibiting formation of dihydropteric acid, used in the folic acid metabolism pathway, by the bacteria. Resistance to sulfonamides is primarily due to export of the antibiotic from the bacteria and a mutation in the target of the antibiotic that reduces the binding affinity of the antibiotic and the target.

In an example, a sample can be taken from a patient for analysis of the presence of antibiotic resistant bacteria. In an example, the sample is sputum, lung lavage, intestinal lavage, nasopharyngeal lavage, saliva, urine, stool, spinal fluid, or blood. In an example, a sample may be any test sample that may contain bacteria. A swab of a surface may be taken to obtain bacteria for analysis.

In various embodiments, bacteria in a patient sample will be isolated by centrifugation. Bacteria in a swab sample are placed in solution. The patient sample or swabbed sample will be incubated with the antibiotic or antibiotics of choice. The bacteria will be lysed or broken open mechanically. In an example, B-PER (Thermo Scientific, Rockford, Ill.) may be used to lyse the bacteria. The lysed cells will be centrifuged to remove cellular debris and the supernatant will be analyzed by an assay disclosed herein.

In an example, one or more of the antibiotics to which the bacteria in the patient sample are not resistant are administered to the patient. In an example, antibiotics to which the bacteria in the patient sample are resistant are not administered to the patient. Antibiotics may be administered to a patient enterally, topically, or parenterally. Enterally includes but is not limited to orally or rectally. Topically includes but is not limited to epicutaneously or mucousally. Parenterally includes but is not limited to intravenously.

Selected reaction monitoring (SRM) analysis of media from bacteria of a known antibiotic resistance allows characterization of the bacterial metabolites of the antibiotics. In selected reaction monitoring (SRM), two mass analyzers act as mass filters to monitor production of a selected precursor ion. The detector provides an intensity value over time by counting the ion matching the selected transition. The two m/z values associated with the precursor and product ion are called a “transition” are written as precursor m/z>product m/z. Multiple SRM reactions can be measured at the same time. This is done by cycling between the different precursor/product transitions. The signal of each transition is recorded based on its elution time.

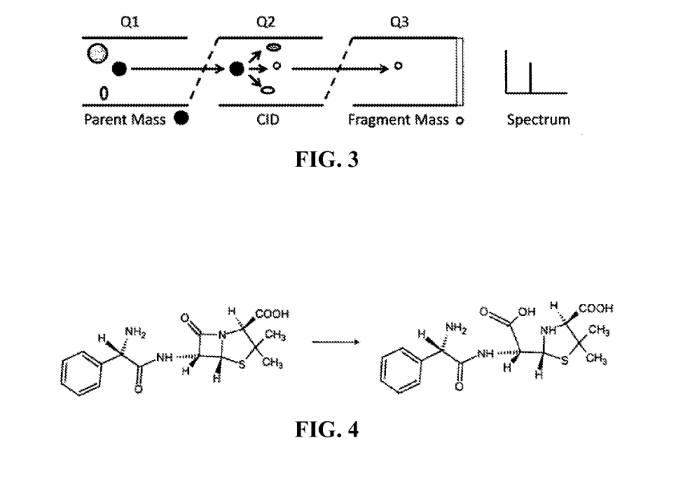

The molecular change in the structure of the antibiotics can be easily monitored through the use of selected reaction monitoring (SRM). (FIG. 3) SRM is a two-stage mass spectrometry technique in which a precursor ion of a particular m/z is isolated during the ionization process, is subjected to collision induced dissociation (CID), and a particular product ion specific to the compound of interest is monitored.

In the first stage, ions of a particular mass-to-charge ratio, the precursor ions, are transmitted through the mass analyzer and the other ions are ejected from the mass analyzer. The precursor ions are transmitted to a collision cell, excited and collide with gas in the mass analyzer. The collisions cause fragmentation of the precursor ions to produce one or more product ions (CID).

In the second stage, the product ions are transmitted to a second mass analyzer. Ions of certain mass-to-charge ratios are selectively transmitted through to the second mass analyzer. The other ions are ejected from the mass analyzer. The selected product ions are detected and monitored as they are transmitted through the second mass analyzer to the detector.

Because m/z values of both precursor and product ions of antibiotics differ from their inactivated forms and from each other, each can be monitored easily by SRM. SRM has two major advantages for analysis. One, SRM is highly selective for an analyte of interest. Two, SRM is extremely sensitive and can quantify samples in the low femtomole level. SRM and high-performance liquid chromatography (HPLC) may be used in combination to monitor a multitude of antibiotics.

In an example, mass spectrometry analysis of media from ampicillin resistant and non-resistant bacteria identified unique spectra for non-resistant samples and resistant samples incubated with ampicillin. Three unique peaks associated with ampicillin metabolites were identified. Other signature spectra were obtained with the media from bacteria resistant to other antibiotics. The metabolites of a particular antibiotic that are created when that antibiotic is exposed to bacteria resistant to the antibiotic will be the same. A similar spectra obtained from a bacteria sample will indicate that bacteria are present that are resistant to that antibiotic because the same metabolites are being formed.

By using the different signature spectra obtained by analysis of resistant and non-resistant bacteria, it is possible to differentiate between resistant and non-resistant bacteria. This method of detection of antibiotic resistance will be effective whenever the bacteria disable the antibiotic causing a change in the molecular weight or mass to charge ratio of the antibiotic. Bacteria may express β-lactamase that cleaves the β-lactam ring of β-lactam antibiotics, converting the antibiotic to an ineffective form. Two hydrogens and one oxygen are added upon cleavage of the β-lactam ring, increasing the molecular weight of ampicillin by 18.02 atomic mass units (amu). The increased molecular weight allows differentiation between active and inactivated ampicillin.

Different signature spectra obtained by analysis of resistant and non-resistant bacteria can also be used to detect bacteria resistant to chloramphenicol. The enzyme that deactivates chloramphenicol, chloramphenicol acetyltransferase (CAT), adds acetyl groups to chloramphenicol from acetyl CoA. A difference of a multiple of 42.02 amu is expected for the inactivated antibiotic.

Advantages of the analysis of the antibiotic by mass spectrometry and the luciferase based ATP-determination assay are (1) The ability to detect antibiotic resistance based on modifications of the antibiotic structure from both single strain bacteria and mixed populations of bacteria. (2) The ability to detect antibiotic resistance from organisms that use different modes of action for resistance. If resistance is due to the organism being able to modify an antibiotic to an inactive form, SRM can be used to detect the change. (3) The ability to detect resistance to multiple antibiotics in a single assay. This is ideal for testing whether a bacterial strain is resistant to multiple antibiotics (superbugs) and also to determine if the same organism is susceptible to any number of antibiotics in the same assay. (4) The ability to detect resistance from very low numbers of resistant bacteria. Therefore, there is not a delay in detection because the bacteria needed to be cultured. (5) The ability to detect antibiotic resistance from very small amounts of sample. As little as one microliter of growth media is needed and a femtomole of antibiotic and its modified products can be detected in a single assay. (6) The assay can be performed in less than an hour. The kinetics of antibiotic modification can also be assayed (penicillins may be hydrolyzed in less than one minute). (7) The assay may be performed with equipment already available and commonly used in clinical settings.

The mass spectrometry assays disclosed herein provides a protocol for performing SRM, a standardized kit (including a mixture of antibiotics of known concentrations and internal standards), a database of all of the m/z values for the antibiotics, metabolites, and values for performing the SRM that would include the m/z values for the antibiotics, metabolites, and all mass spectrometry parameters.

Rapid determination of antibiotic resistance is possible by the disclosed mass spectrometry and a luciferase based ATP-determination assay. These methods may be utilized in a microfluidics device to produce a simple, fast, relatively inexpensive diagnostic tool for use in hospitals and clinics. A microfluidic device has one or more channels that may be less than 1 millimeter. The volume of fluids and reagents required may be small. The microfluidic device may be composed of silica glass or other suitable material.

Microfluidics devices for performing the assays disclosed herein would have high sensitivity, provide a rapid method for detection, and decrease costs. In an example, a microfluidic device for detecting antibiotic resistance may comprise a solid substrate comprising a sample entry port, buffer in a buffer chamber, and an antibiotic chamber, wherein upon adding a sample to the microfluidic device, the sample, the buffer, and the antibiotic would then mix together, providing an output sample to be injected for analysis of the molecular weight of the antibiotic. The cells may be lysed in the microfluidic device prior to obtaining an output sample. In an example, a chamber may be present to separate the antibiotic based upon molecular weight. In an example, a chamber may be present to separate the medicinal compound based upon molecular weight. In an example, a chamber may be present to separate the chemotherapeutic based upon molecular weight. In an example, the output sample may be analyzed by HPLC. In an example, the output sample may be analyzed by mass spectrometry.

The mass spectrometry assays disclosed herein may be used to detect the modification of any medicinal compound. As long as the modification affects the mass to charge ratio of the medicinal compound, the mass spectrometry assay can detect the modification.

In an embodiment, the mass spectrometry and growth assays may be used to detect metabolism of a medicinal compound. The mass spectrometry assay may detect modifications of a medicinal compound, the modifications including, but not limited to glucoronidation, sulfation, oxidation, hydroxylation, dealkylation, and hydrolysis. Studies of metabolism of a medicinal compound by the mass spectrometry assay disclosed herein can provide information upon the time frame in which the medicinal compound is metabolized in the sample or whether the medicinal compound is being metabolized incorrectly. In an embodiment, incorrect metabolism may include but is not limited to, metabolism that occurs when not expected, metabolism that does not occur when expected, and metabolism that occurs by a different modification than expected. Organs that have metabolic functions include but are not limited to the liver, gastrointestinal tract, kidneys, and lungs. Cytochrome P450 enzymes may be involved in metabolism. Cytochrome P450 enzymes catalyze aromatic hydroxylation, aliphatic hydroxylation, N—, O—, and S-dealkylation, N-hydroxylation, N-oxidation, sulfoxidation, deamination, and dehalogenation. Other enzymes that may be involved in metabolism of medicinal compounds include but are not limited to esterases, amidases, proteases, and transferases. Modification of a medicinal compound may decrease the toxicity of the medicinal compound. The mass spectrometry assays disclosed herein can be applied to the testing of a sample for the modification of a medicinal compound by the identification of metabolites by mass spectrometry. The growth assays disclosed herein can be used to detect the growth of cells in the presence of medicinal compounds individually or in combinations to detect growth or lack of growth in the presence of the medicinal compounds.

In an embodiment, the mass spectrometry and growth assays may be used to detect whether a parasite is resistant to a medicinal compound. The assay can detect whether the medicinal compound is metabolized to a non-toxic metabolite by the parasite. Parasites that may exhibit drug resistance include but are not limited to Plasmodium, Giardia, Entamoeba, Trichomonas, and Trypanosoma. Medicinal compounds that may be used to treat parasites include but are not limited to sulfadoxine, dapsone, pyrimethamine, proguanil, chloroquine, mefloquine, quinine, atovaquone, and artemisinins The mass spectrometry assays disclosed herein can be applied to the testing of parasites for their resistance to medicinal compounds by the identification of drug metabolites indicative of resistance by mass spectrometry. The growth assays disclosed herein can be used to detect the growth of parasites in the presence of medicinal compounds individually or in combinations to detect resistance that does not involve the modification of the agents.

In an embodiment, the mass spectrometry and growth assays may be used to detect the modification of a medicinal compound administered to a patient to treat cancer (a chemotherapeutic). The approaches to cancer chemotherapy have evolved into increasing complex and customized protocols in response to the cancer and more recently, the individual patient. Cancer cells can be inherently resistant to the chemotherapeutics or can acquire resistance through several means including: expression of transporters that eject the chemotherapeutic drugs, insensitivity to drug induced apoptosis, mutations in drug targets and the chemical modification by cellular enzymes that inactivate the drugs and/or target them for export. The issue of single and multiply drug resistant tumors has become even more serious with the recognition of the presence of tumorigenic stem cells in a variety of tumors. This has raised the question if the innate resistance of normal stem cells to radiation and toxins extends to tumorigenic stem cells and may be responsible for the failure of some chemotherapies. The mass spectrometry assays disclosed herein can be applied to the testing of tumor cells for their resistance to chemotherapeutics by the identification of drug metabolites indicative of resistance by mass spectrometry. The growth assays disclosed herein can be used to detect the growth of tumor cells in the presence of chemotherapeutic agents individually or in combinations to detect resistance that does not involve the modification of the agents.

Chemotherapeutic drugs such as cyclophosphamide, which is commonly used to treat breast cancer, can be inactivated by isoforms of aldehyde dehydrogenase (ALDH). Breast cancer cells have elevated levels of the ALDH isoform 3A1 when compared to normal breast, while metastatic tumors resistant to cyclophosphamide treatment showed elevation of ALDH1A1. Alkylating agents like cyclophosphamide and other chemotherapeutic agents such as cisplatin and other platinum containing compounds may also be detoxified by glutathione and the action of glutathione S-transferase (GST). Cells with high levels of GST inactivate these drugs by conjugating them with GST. Another example is the actions of cytochrome p450 enzymes on the family of taxane anti-tumor drugs of which Paclitaxel (TAXOL®) is the prototype. Several of these mechanisms of drug modification work on entire classes of drugs leading to multiply drug resistant tumors.

In an example, a microfluidic device for detecting antibiotic resistance may comprise a solid substrate comprising a sample entry port, buffer in a buffer chamber, and a chemotherapeutic chamber, wherein upon adding a sample to the microfluidic device, the sample, the buffer, and the chemotherapeutic would then mix together, providing an output sample to be injected for analysis of the molecular weight of the chemotherapeutic. The cells may be lysed in the microfluidic device prior to obtaining an output sample. In an example, a chamber may be present to separate the chemotherapeutic based upon molecular weight. In an example, a chamber may be present to separate the chemotherapeutic based upon molecular weight. In an example, the output sample may be analyzed by HPLC. In an example, the output sample may be analyzed by mass spectrometry.

The following examples are included to demonstrate preferred embodiments of the present disclosure. It should be appreciated by those of skill in the art that the techniques disclosed in the examples which follow represent techniques discovered by the inventors to function well and thus can be considered to constitute preferred modes for its practice. However, those of skill in the art should, in light of the present disclosure, appreciate that many changes can be made in the specific embodiments which are disclosed and still obtain a like or similar result without departing from the spirit or scope of the disclosure. The following Examples are offered by way of illustration and not by way of limitation.

### EXAMPLE 1

Ampicillin resistant E. coli C600 cells harboring pUC18 and non-resistant E. coli RR1 were grown in M9 minimal media with casamino acids and ampicillin and collected by centrifugation at 3000 rpm at 4° C. for ten minutes. The supernatant was removed and filtered through a 0.45 micron filter and stored frozen before mass spectroscopic analysis. The breakdown products were detected, samples were taken from a resistant culture every hour for eight hours and after overnight growth. These samples were prepared as before, and then filtered with a 0.22 μm syringe filter. These samples were also frozen before mass spectroscopic analysis.

Breakdown products were detected in a more diverse bacterial culture, fresh mouse fecal pellets. The mouse fecal pellets were homogenized in M9 minimal media and ampicillin and serially diluted to 1/10⁻⁶. A mid-log phase culture of the resistant strain (pUC18) was made and 0, 5, 10, and 100 μL of this culture were added to 50 and 100 μL of the 10⁻⁶ mouse pellet dilutions. These were grown for four hours, centrifuged and filtered as described above, and analyzed by mass spectroscopic analysis.

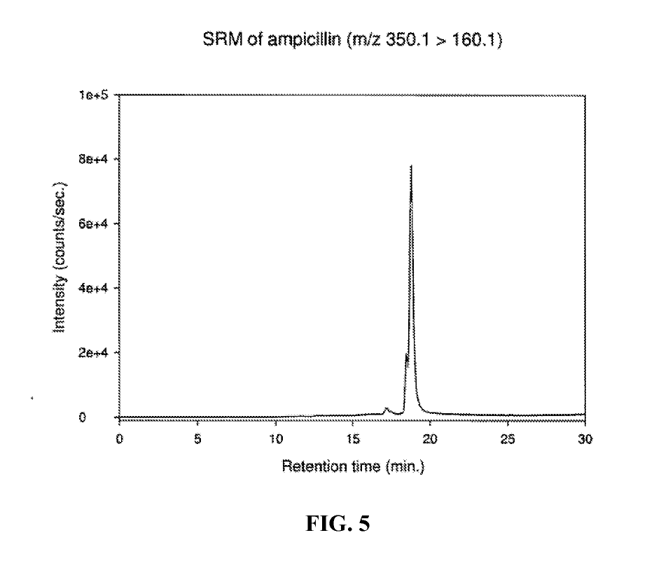

Ampicillin is degraded by β-lactamase to yield the biologically inactive ampicillin penicilloic acid (FIG. 4) through the cleavage of the β-lactam ring. The high performance liquid chromatography mass spectrometric spectra by SRM of ampicillin is shown in FIG. 5.

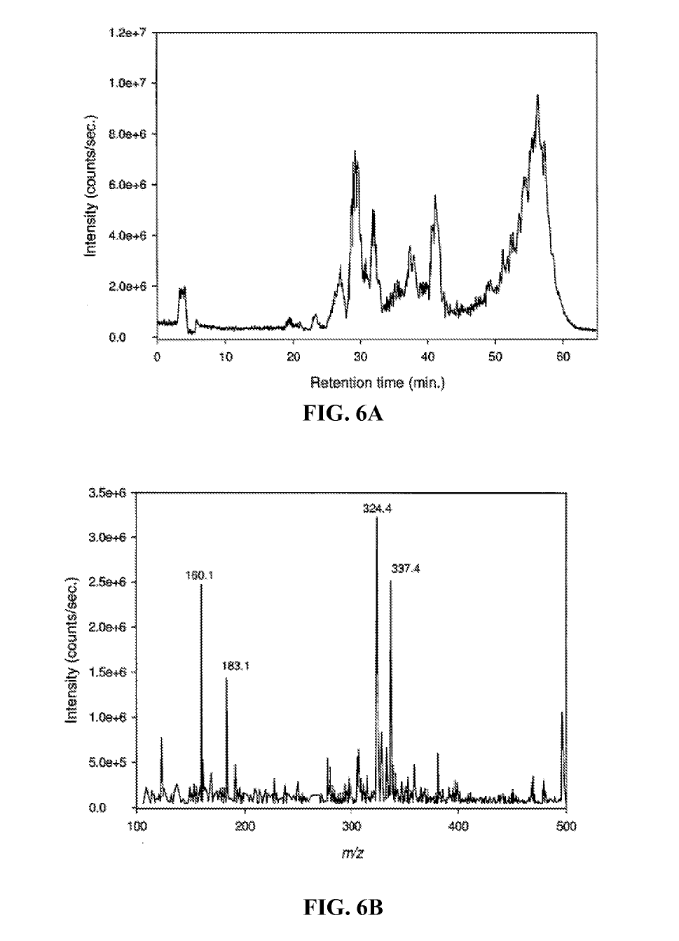

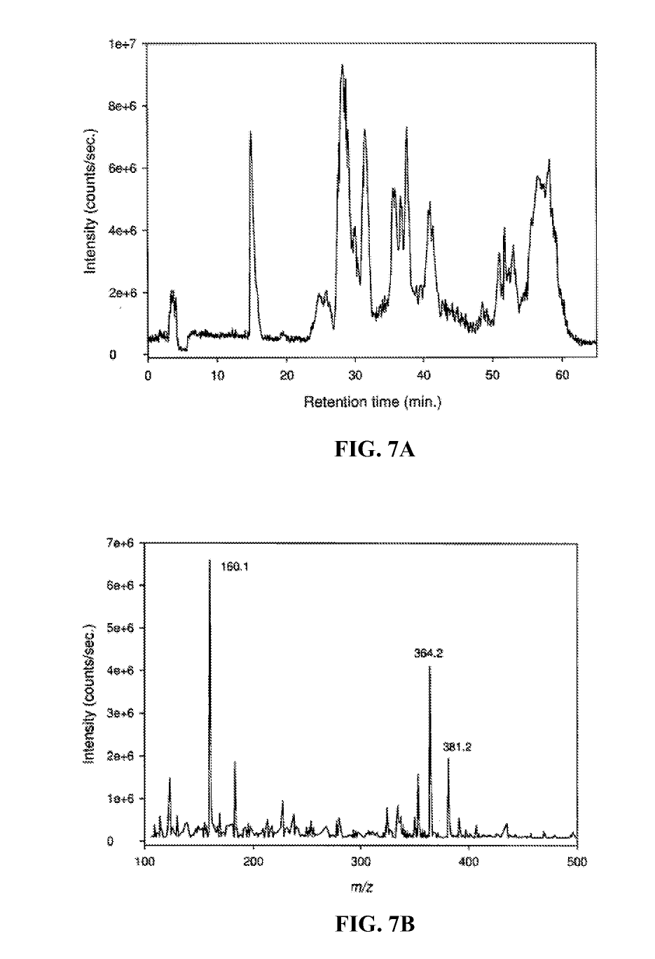

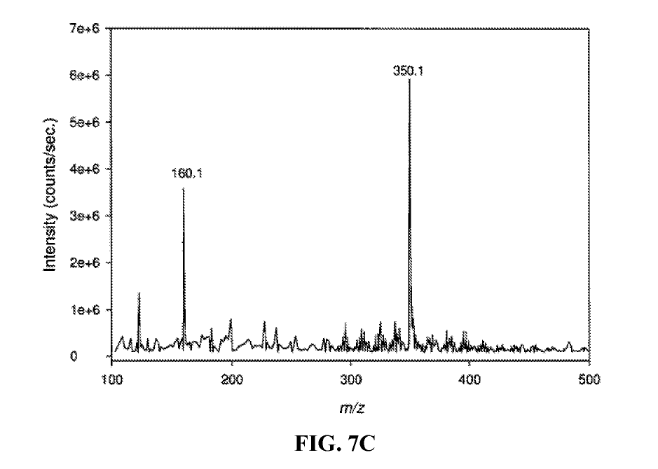

The results of the mass spectrometric analysis indicate that a signature spectrum of ampicillin breakdown products can be seen in culture supernatants of the ampicillin resistant bacteria. Peaks at 183.1, 324.4, and 337.4 amu present in the sample of media from ampicillin resistant E. coli (FIG. 6A and FIG. 6B) are completely absent in the non-resistant sample (FIG. 7A, FIG. 7B, and FIG. 7C).

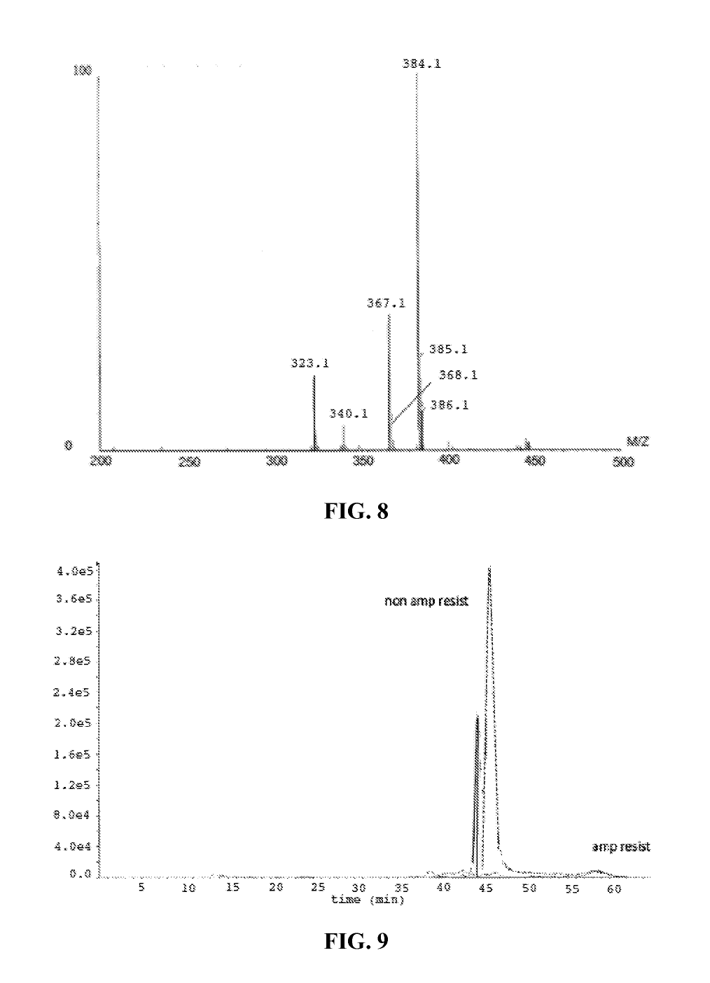

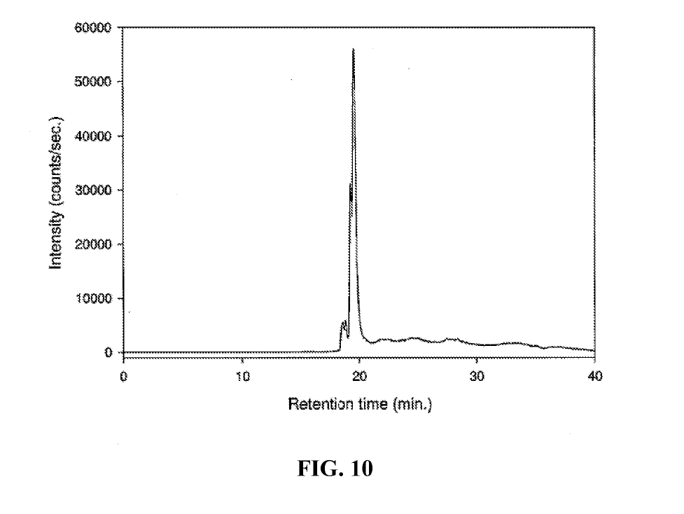

Ampicillin in solution naturally degrades into several products (FIG. 8) when in solution. These breakdown products however, are distinct from both those when ampicillin is incubated with β-lactamase, and those found in the culture supernatants of ampicillin resistant bacteria. (FIG. 9) The products seen in the media from antibiotic resistant bacteria are likely from the initial cleavage of the β-lactam ring by β-lactamase, and the subsequent changes the products undergo, since these two spectra are different. Culture supernatants from the growth of E. coli contain a complex mixture of organic compounds and bacterial enzymes that could lead to structural changes and chemical modifications of the β-lactam ring cleaved ampicillin. Ampicillin incubated with purified β-lactamase (FIG. 10) also showed a distinct spectrum.

### EXAMPLE 2

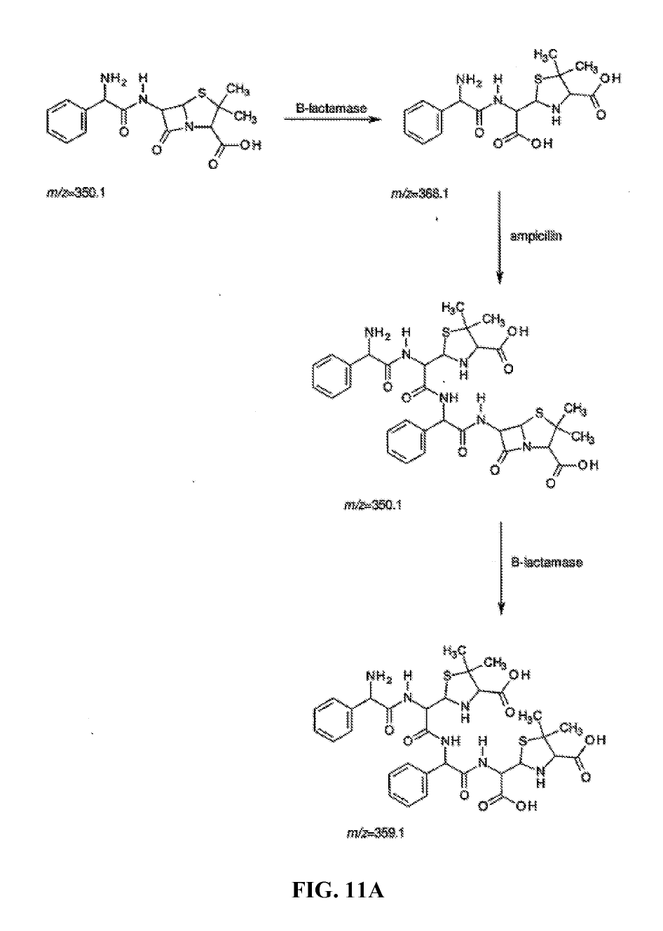

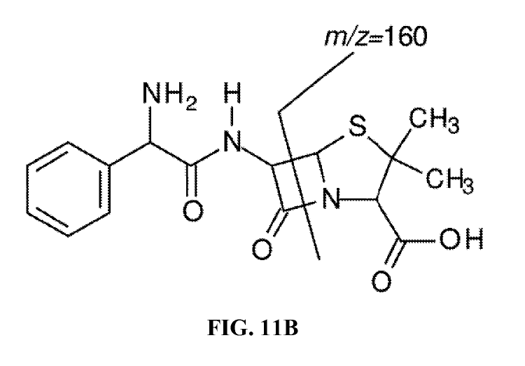

SRM was used to monitor the hydrolysis of ampicillin by a β-lactamase positive strain of E. coli. However, the carboxylic acid formed via β-lactam ring opening is highly reactive and reacts with the amine group of another ampicillin molecule through an electrophilic reaction. This dimer can then be hydrolyzed again by β-lactamase. The reaction is illustrated in FIG. 11A. The m/z for the precursor of ampicillin is 350.1 and a product ion of m/z=160.1. For the hydrolyzed ampicillin, the dimer has an m/z precursor of 359.1 (doubly charged ion) and product ion of m/z=160.1. The product ion of m/z=160.1 is depicted in FIG. 11B.

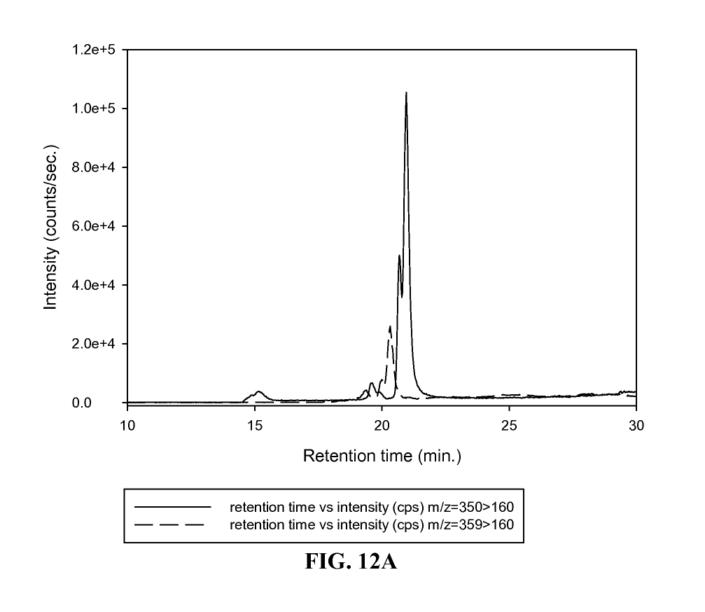

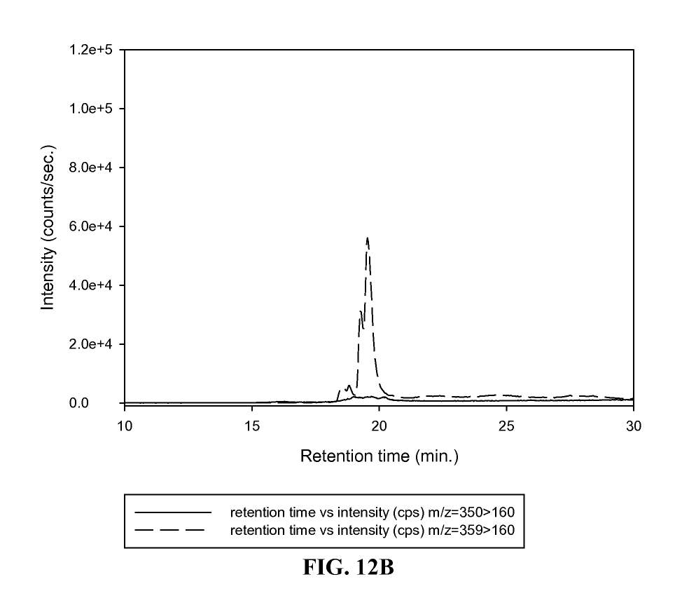

After only minutes all ampicillin is hydrolyzed by β-lactamase producing E. coli. (FIGS. 12A and 12B). The peak labeled amp in FIG. 12A represents the SRM of ampicillin (m/z 350.1 to 160.1). The peak labeled hyd amp in FIG. 12B represents the formation of the hydrolyzed ampicillin (m/z 359.1 to 160.1). Also, a shift in the retention time of the SRM transition 350.1>160.1 is that of the formation of a hydrolyzed byproduct of ampicillin that undergoes a rearrangement to form a byproduct with the same transition state. However, the two can be resolved by the retention time in the HPLC trace.

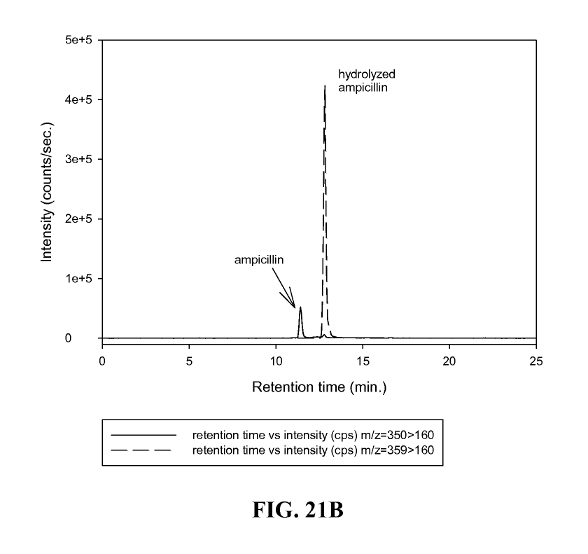

This study was repeated for ampicillin with the following modifications: a 10 cm×75 μm column containing Agilent Zorbax SB-C18 5 μm packing is used instead of Dionex PepMap100 C18 5 μm packing Several parameters in the mass spectrometer were also made. The collision energy (CE), entrance (EP) and collision exit potentials (CXP), and declustering potentials (DP) were optimized for both ampicillin and its hydrolyzed form. The optimized values for ampicillin were CE=19 (at m/z=350>160), EP=11, CXP=11, DP=60. The optimized values for the hydrolyzed ampicillin product were CE=25 (at m/z=359>160), EP=10, CXP=8, DP=60. The elution order of ampicillin and hydrolyzed ampicillin are switched and the peaks are much better resolved (FIG. 21A and FIG. 21B) as compared to FIG. 12A and FIG. 12B. The sensitivity also increased more than an order of magnitude.

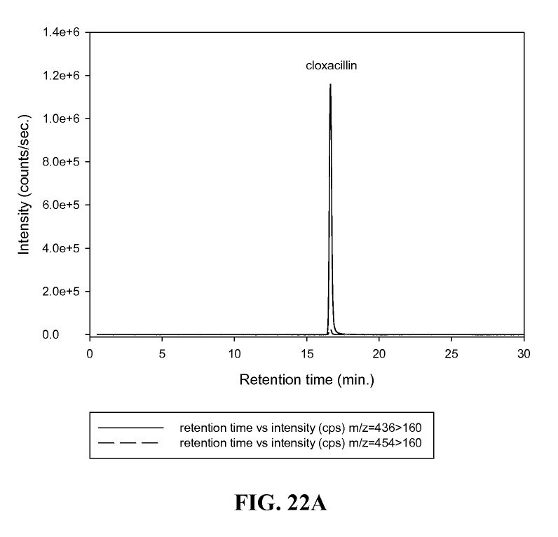

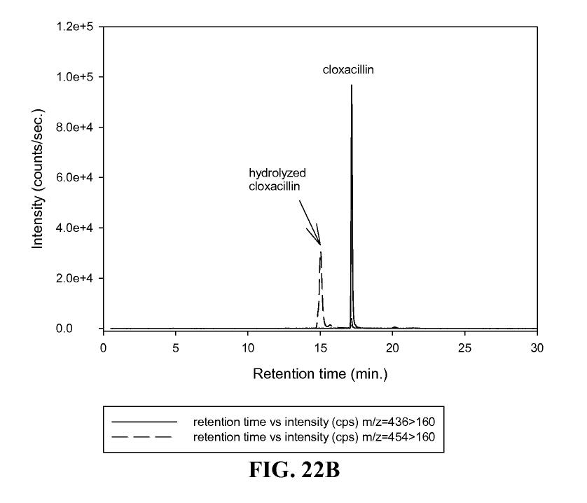

An additional study was performed using cloxacillin instead of ampicillin (FIG. 22A and FIG. 22B) with the following modifications: a 10 cm×75 μm column containing Agilent Zorbax SB-C18 5 μm packing is used. The collision energy (CE), entrance (EP) and collision exit potentials (CXP), and declustering potentials (DP) were optimized for both cloxacillin and its hydrolyzed form. The optimized values for cloxacillin were CE=22 (at m/z=436>160), EP=8, CXP=8, DP=60. The optimized values for the hydrolyzed ampicillin product were CE=22 (at m/z=454>160), EP=8, CXP=8, DP=60. Cloxacillin is also hydrolyzed by β-lactamase, but usually at a slower rate. However, hydrolyzed cloxacillin can be clearly identified even after several minutes of exposure to β-lactamase as illustrated in FIG. 22B.

### EXAMPLE 3

E. coli C600 cells harboring the chloramphenicol resistance plasmid pACYC184 and non-resistant E. coli RR1 were grown in M9 minimal media with casamino acids and chloramphenicol and collected by centrifugation at 3000 rpm at 4° C. for ten minutes. The supernatant was removed and filtered through a 0.45 micron filter and stored frozen before mass spectroscopic analysis. The breakdown products were detected, samples were taken from a resistant culture every hour for eight hours and after overnight growth. These samples were prepared as before, and then filtered with a 0.22 μm syringe filter. These samples were also frozen before mass spectroscopic analysis.

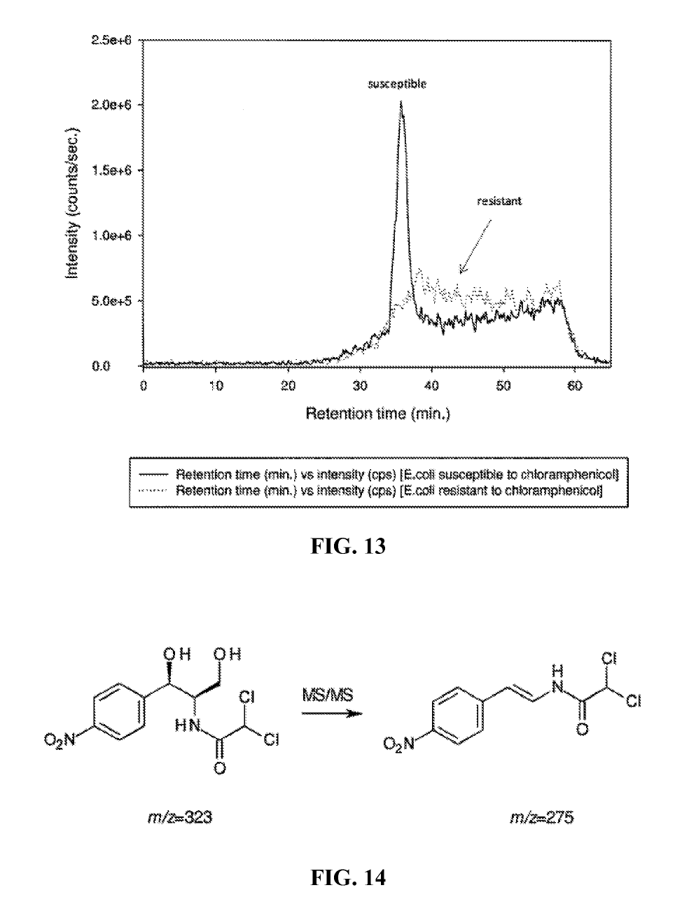

Chloramphenicol resistant bacteria yielded analogous results. The M9 media containing chloramphenicol was compared to M9 media supernatants from chloramphenicol resistant bacteria (FIG. 13) to show the metabolism of chloramphenicol by the bacteria containing the resistance plasmid pACYC 184. A “fingerprint” can be prepared for chloramphenicol modified metabolites.

For chloramphenicol, M9 broth with chloramphenicol shows a peak but the media in which chloramphenicol resistant bacteria were grown shows no peak for chloramphenicol (FIG. 13). The m/z ranges from 323 to 325 amu, encompasses chloramphenicol but excludes its degradation products.

### EXAMPLE 4

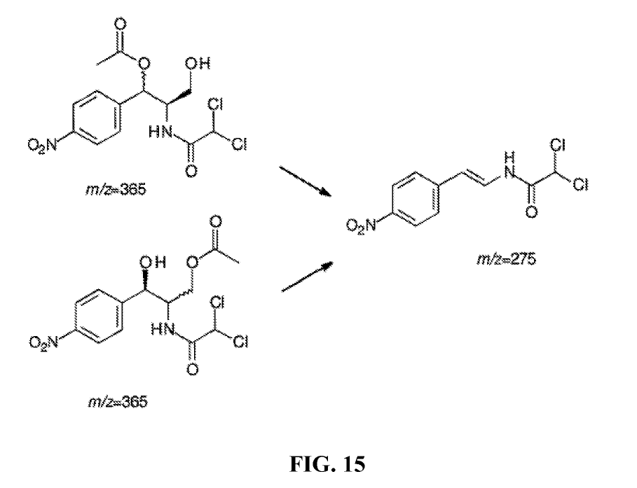

SRM may be used to monitor the decrease in chloramphenicol and increase in acetylated chloramphenicol when chloramphenicol is subjected to chloramphenicol resistant bacteria. Chloramphenicol has a protonated m/z value of 323.0 for the precursor ion and a CID product ion of m/z=275.0. This corresponds to the loss of the hydroxyls and methylene group during CID (FIG. 14). There are two possible acetylation sites for chloramphenicol. However, both have the same m/z value (365.0) for the precursor ion and the same CID product ion (275.0) and can therefore be monitored concurrently in the same experiment (FIG. 15).

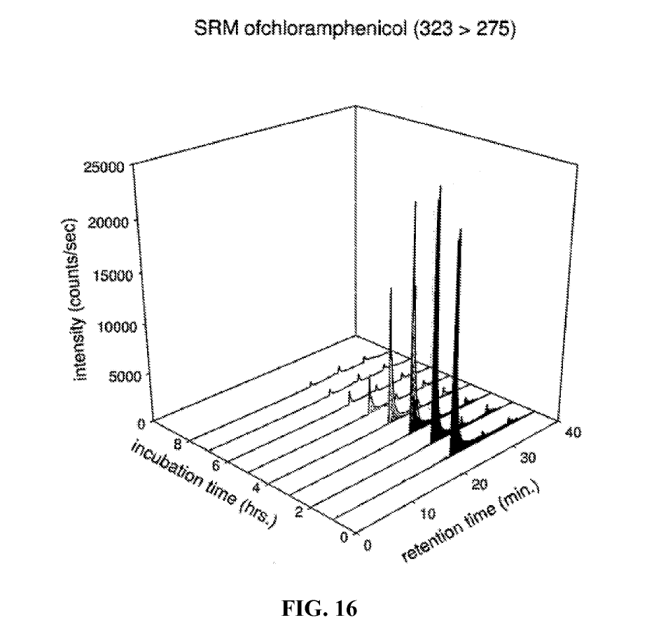

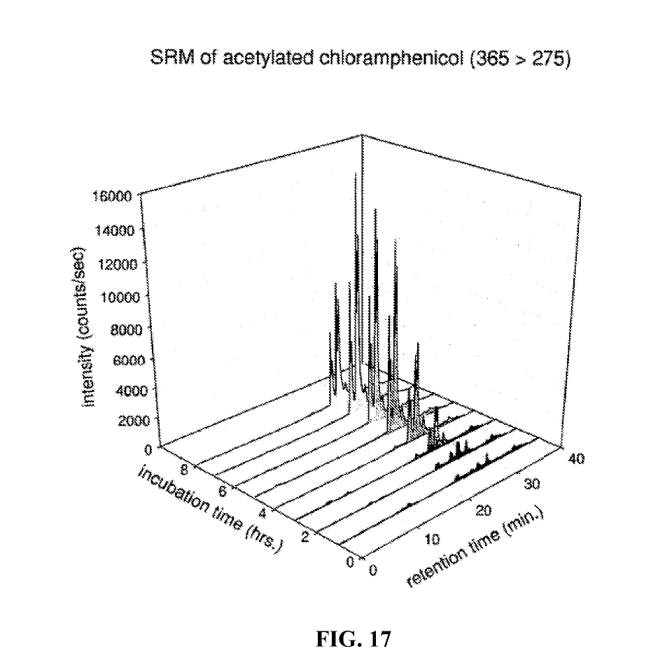

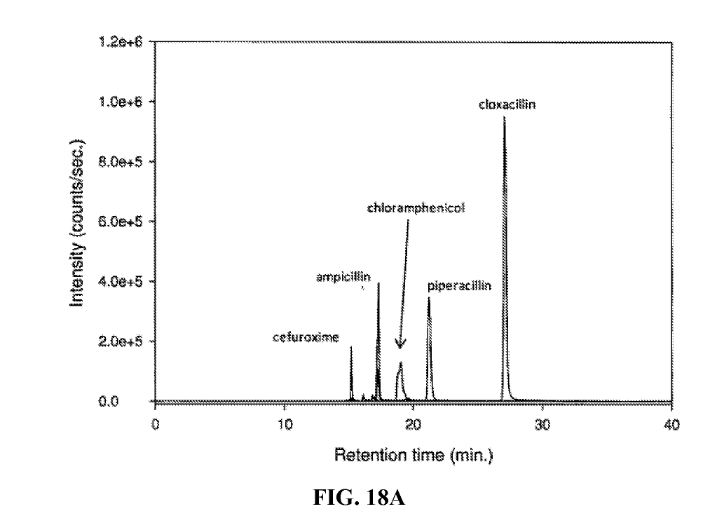

To determine if SRM can be used to identify and monitor the acetylation of chloramphenicol by a chloramphenicol resistant strain of bacteria, a known strain of chloramphenicol resistant E. coli was grown in a broth containing chloramphenicol. Samples of the broth were obtained at 1 hr intervals and analyzed by SRM. FIG. 16 illustrates the reduction of chloramphenicol in the broth over time. FIG. 17 illustrates the formation of the inactive acetylchloramphenicol over the same period of time. In each case, the samples were diluted 1:1,000 and less than 5 μL of each diluted sample was used for analysis.

### EXAMPLE 5

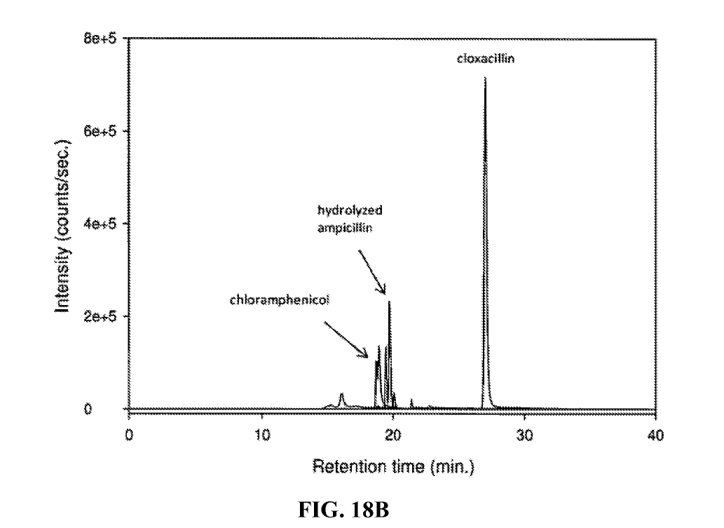

SRM may be used to screen a variety of antibiotics at one time. Five different antibiotics were added together and then subjected to ⊕-lactamase. The five antibiotics were ampicillin, cloxacillin, cephalexin, piperacillin, and chloramphenicol. Of these, cloxacillin is resistant to β-lactamase and chloramphenicol is not affected by β-lactamase. FIG. 18A depicts the 5 antibiotic mixture and FIG. 18B is the 5 antibiotic mixture after being subjected to β-lactamase. The reaction time was less than 5 minutes. Ampicillin, piperacillin, and cephalexin were completely hydrolyzed while cloxacillin and chloramphenicol remained unaffected.

### EXAMPLE 6

Since metabolism of the antibiotic is not the only method of antibiotic resistance, a luciferase based ATP assay was also used to detect antibiotic resistance. Those bacteria that pump antibiotic out of the cell, do not allow it to enter, or have mutations in the target of the antibiotic can still grow in the presence of the antibiotic but will not show any antibiotic metabolites with mass spectroscopic analysis. To detect these modes of antibiotic resistance, a measurement of total bacterial ATP was used. In an embodiment, the luciferase based ATP assay may be used to confirm growth of the bacteria in mass spectrometry assays. Other ATP assays may be used instead of the luciferase based ATP assay.

Samples of bacteria harboring the plasmids pUC18 and pACYC184 were grown to mid-log phase, and then diluted serially to 1/10⁻⁸ in sterile water. Both plasmids are in the same E. coli strain C600. The bacteria in these dilutions were collected by centrifugation, then lysed with B-PER (Thermo Scientific, Rockford, Ill.). The lysate was clarified by centrifugation and the supernatant used in the ATP assay. A Veritas luminometer with an inject function and luciferase ATP bioluminescent assay kit (Sigma-Aldrich, St Louis, Mo.) were used to measure ATP levels in each sample using white and black 96-well plates containing 50 μL of each sample. 50 μL of the 1/25 assay mix in dilution buffer was injected into each well directly before the measurement was taken. This was repeated with the fully concentrated assay mix for dilutions 1/10⁻⁵ through 1/10⁻⁸ dilutions of the lysed bacteria. An ATP standard curve was produced with ATP dilutions between 2×10⁻³ and 2×10⁻¹² M prior to each use of the luminometer.

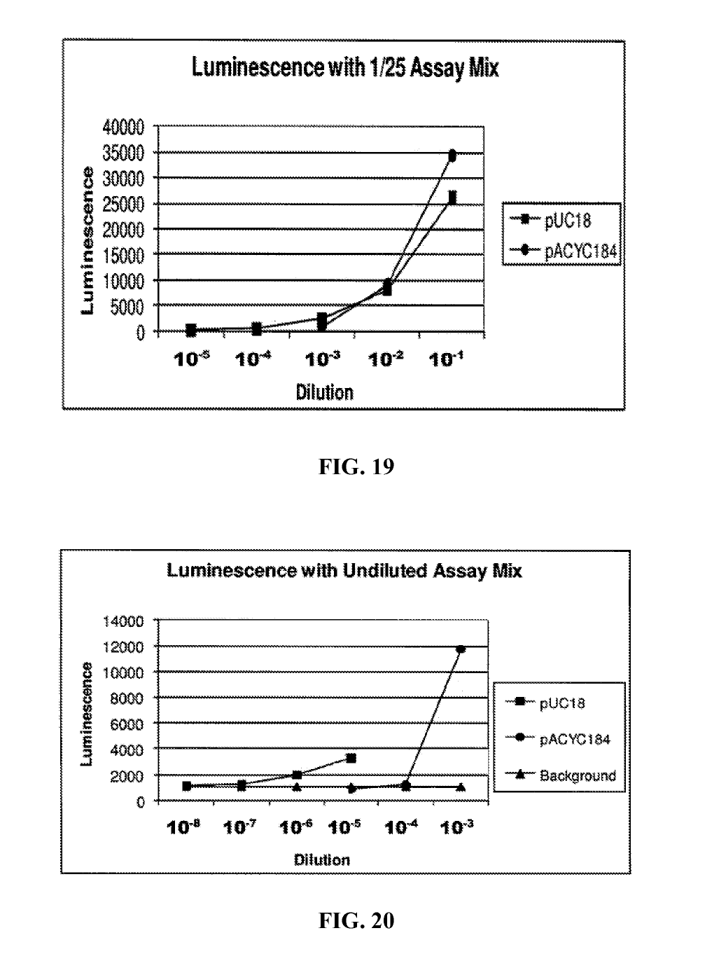

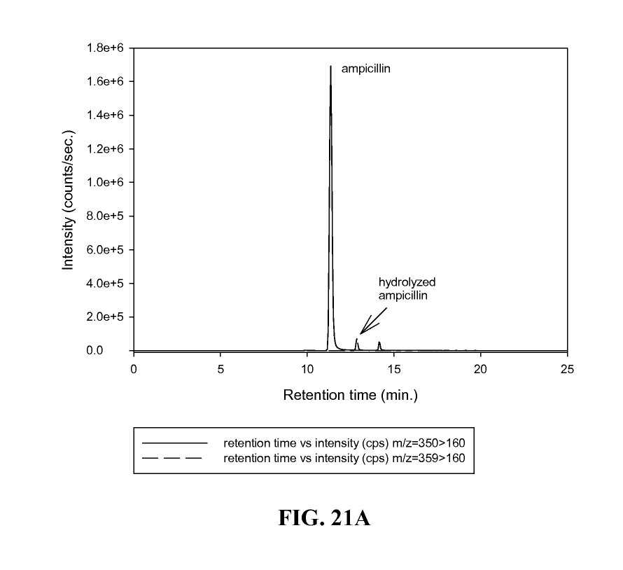

A 1/25 dilution of the assay mix in dilution buffer (FIG. 19) proved to be too great a dilution to be effective on the scale that was desired. The 1/25 dilution of assay mix was only able to detect down to 2×10⁻¹⁰ M ATP, whereas the undiluted mix could easily detect 2×10⁻¹⁵ M ATP. The luciferase assay proved to be highly sensitive when the undiluted assay mix was used (FIG. 20). The assay could detect between one and ten bacteria with the pUC 18 plasmid, but required at least 1100 bacteria with pACYC184 to detect the growth.

FIG. 19, FIG. 20, and Table 1 show the results of the luciferase based ATP determination assay. The 1/25 dilute assay mix (FIG. 19) was much less effective than the undiluted version (FIG. 20). With the undiluted assay mix, between 1-10 colony forming units (CFU) of the pUC18 containing strand were able to be detected, while about 1100 CFU were detected in E. coli harboring pACYC184. The detection of 1000 bacteria represents an extremely sensitive approach to antimicrobial resistance. Table 1 shows the number of observed bacteria at each dilution. TMTC indicates a plate count of over 300 bacteria.

TABLE 1
Number of Bacteria Observed at Each Dilution

| Dilution | pUC18(CFU) | pACYC184(CFU) |
|---|---|---|
| 10⁻¹ | TMTC | TMTC |
| 10⁻² | TMTC | TMTC |
| 10⁻³ | TMTC | TMTC |
| 10⁻⁴ | 190 | TMTC |
| 10⁻⁵ | 12 | 304 |
| 10⁻⁶ | 1 | 44 |
| 10⁻⁷ | 0 | 9 |
| 10⁻⁸ | 0 | 0 |

### EXAMPLE 7

The mass spectrometry assays disclosed herein may be used to detect the modification of any medicinal compound. In an embodiment, the mass spectrometry assays may be used to detect the modification of a chemotherapeutic that may be administered to a patient to treat cancer.

In designing the ideal chemotherapeutic regimen, tumor cells should be tested for drug resistance and sensitivity. Lab tests can be performed from biopsy, blood, bone marrow, or malignant fluid. Tumor or tumor cells will be collected and placed in growth and stabilization media and transported to the laboratory. There the samples will be processed to achieve a single cell suspension and the sample will be divided into 2 parts, one for mass spectrometry analysis and one for analysis of growth. For mass spectrometry, the cells will be divided in cultures and chemotherapeutic drugs known to be appropriate for the tumor type will be added alone or in predetermined combinations. The cells will be cultured in the presence and absence of drugs for 4 hours, then lysed and incubated for 30 minutes. The lysates will be clarified and analyzed for the presence of the parent and modified chemotherapeutic drugs by the LC MS/MS methods disclosed herein. The second aliquot of cells will be aliquoted into growth chambers and suspended in growth medium alone or with the addition of individual chemotherapeutic drugs and allowed to grow for 6 hours. The cells are then lysed and the amount of ATP measured in each sample using the luciferase based ATP assay. Increases in ATP above baseline are a measure of cell growth and will provide a panel of drug resistance or susceptibility information on that tumor. Other ATP assays may be used in place of the luciferase ATP assay.

Variations and modifications to the preferred embodiments of the disclosure described herein will be apparent to those skilled in the art. It is intended that such variations and modifications may be made without departing from the scope of the disclosure and without diminishing its attendant advantages.

All of the compositions and methods disclosed and claimed herein can be made and executed without undue experimentation in light of the present disclosure. While the compositions and methods of this disclosure have been described in terms of preferred embodiments, it will be apparent to those of skill in the art that variations may be applied to the compositions and methods and in the steps or in the sequence of steps of the methods described herein without departing from the concept, spirit and scope of the disclosure. More specifically, it will be apparent that certain agents which are both chemically and physiologically related may be substituted for the agents described herein while the same or similar results would be achieved. All such similar substitutes and modifications apparent to those skilled in the art are deemed to be within the spirit, scope and concept of the disclosure as defined by the appended claims.

1. A method of identifying a drug resistance profile of a sample comprising:
exposing a sample to at least one drug;
conducting mass spectrometry on at least one drug after exposure to the sample and analyzing mass to charge ratio of ions from the at least one drug; and
detecting the presence of one or more ions indicative of the drug and/or a metabolite of the drug.

2. The method of claim 1, wherein the mass spectrometry is electrospray mass spectrometry.

3. The method of claim 1, wherein the mass spectrometry is selected reaction monitoring.

4. The method of claim 1, wherein the sample comprises at least one antibiotic resistant bacterium.

5. The method of claim 1, wherein the sample comprises tumor cells.

6. The method of claim 1, wherein the sample is exposed to more than one drug.

7. The method of claim 1, wherein ions from more than one drug are detected.

8. The method of claim 1, wherein the drug is an antibiotic.

9. The method of claim 8, wherein the antibiotic is a member of the penicillin or cephalosporin family of antibiotics.

10. The method of claim 9, wherein a penicillin is selected from ampicillin, amoxicillin, azlocillin, bacampicillin, cefixime, carbenicillin, methicillin, cloxacillin, 6-APA, piperacillin, pivmecillinam, penicillin V, monolactam, aztreonam, mecillinam, imipenem, or meropenem.

11. The method of claim 9, wherein a cephalosporin is selected from cefoperazone, latamoxef, cephapirin, cefazolin, cefaclor, ceftibuten, ceftizoxime, cefotetan, cefuroxime, cefprozil, ceftazidime, cephaloglycine, cephaloridine, nitrocephine, cefatoxime, ceftiofur, cephapyrine, cefepime, cefpirome, cefadroxil, cefamandole, cefoxitin, cefpodoxime, ceftriaxone, cephalexin, cephazoline, cephradine or 7-ACA.

12. The method of claim 1, wherein the drug is a chemotherapeutic agent.

13. The method of claim 12, wherein the chemotherapeutic agent is cyclophosphamide or paclitaxel.

14. The method of claim 1, wherein the time between exposing a sample to a drug and conducting mass spectrometry is less than 60 minutes.

15. The method of claim 1, wherein one or more ions are indicative of a metabolite of the drug.

16. The method of claim 1, wherein the metabolite is a glucoronidation, sulfation, oxidation, hydroxylation, dealkylation, or hydrolysis product.

17. The method of claim 11, wherein the metabolite is a hydrolysis product.

18. The method of claim 1, further comprising administering a drug to a patient, from which the sample was obtained, that is not inactivated upon exposure to the sample.

19. The method of claim 1, wherein the sample is selected from the group consisting of sputum, saliva, urine, stool, spinal fluid, lung lavage, intestinal lavage, nasopharyngeal lavage and blood.
